SEQUENCE LISTING (41 sequences)

SEQ ID NO: 1 - protein, 10 aa, Artificial Sequence
  Description of Artificial Sequence Synthesized peptide
  MISC_FEATURE (3)..(3): Xaa is an aromatic amino acid Phe, Trp or Tyr
  MISC_FEATURE (6)..(6): Xaa is an aromatic amino acid Phe, Trp or Tyr
CGXGRXSPPC

SEQ ID NO: 2 - protein, 10 aa, Artificial Sequence
  Description of Artificial Sequence Synthesized peptide
  MISC_FEATURE (3)..(3): Xaa is any aromatic amino acid Phe, Trp, or Tyr
  MISC_FEATURE (6)..(6): Xaa is any aromatic amino acid Phe, Trp, or Tyr
CGXGRXQPPC

SEQ ID NO: 3 - protein, 10 aa, Artificial Sequence
  Description of Artificial Sequence Synthesized peptide
  MISC_FEATURE (3)..(3): Xaa is any amino acid
  MISC_FEATURE (7)..(9): Xaa is any amino acid
CRXYGPXXXC

SEQ ID NO: 4 - protein, 10 aa, Artificial Sequence
  Description of Artificial Sequence Synthesized peptide
CGYGRFSPPC

SEQ ID NO: 5 - protein, 10 aa, Artificial Sequence
  Description of Artificial Sequence Synthesized peptide
CGWGRYSPPC

SEQ ID NO: 6 - protein, 10 aa, Artificial Sequence
  Description of Artificial Sequence Synthesized peptide
CGFGRWQPPC

SEQ ID NO: 7 - protein, 10 aa, Artificial Sequence
  Description of Artificial Sequence Synthesized peptide
CRVYGPYLLC

SEQ ID NO: 8 - protein, 10 aa, Artificial Sequence
  Description of Artificial Sequence Synthesized peptide
CRWYGPILWC

SEQ ID NO: 9 - protein, 10 aa, Artificial Sequence
  Description of Artificial Sequence Synthesized peptide
CRWYGPWALC

SEQ ID NO: 10 - protein, 10 aa, Artificial Sequence
  Description of Artificial Sequence Synthesized peptide
CRWYGPWVWC

SEQ ID NO: 11 - protein, 10 aa, Artificial Sequence
  Description of Artificial Sequence Synthesized peptide
CRFYGAWLLC

SEQ ID NO: 12 - protein, 10 aa, Artificial Sequence
  Description of Artificial Sequence Synthesized peptide
CRHYGPFSIC

SEQ ID NO: 13 - protein, 10 aa, Artificial Sequence
  Description of Artificial Sequence Synthesized peptide
CRRYGPFMVC

SEQ ID NO: 14 - protein, 10 aa, Artificial Sequence
  Description of Artificial Sequence Synthesized peptide
CRTYGWWVVC

SEQ ID NO: 15 - protein, 10 aa, Artificial Sequence
  Description of Artificial Sequence Synthesized peptide
CRYYGWLTVC

SEQ ID NO: 16 - protein, 10 aa, Artificial Sequence
  Description of Artificial Sequence Synthesized peptide
CKWYGLFQLC

SEQ ID NO: 17 - protein, 10 aa, Artificial Sequence
  Description of Artificial Sequence Synthesized peptide
CHSYGPFVVC

SEQ ID NO: 18 - protein, 10 aa, Artificial Sequence
  Description of Artificial Sequence Synthesized peptide
CNWYGWFRVC

SEQ ID NO: 19 - protein, 10 aa, Artificial Sequence
  Description of Artificial Sequence Synthesized peptide
CTTHWGFTLC

SEQ ID NO: 20 - protein, 18 aa, Artificial Sequence
  Description of Artificial Sequence Synthesized peptide
ADGACILWMDDGWCGAAG

SEQ ID NO: 21 - protein, 18 aa, Artificial Sequence
  Description of Artificial Sequence Synthesized peptide
TTPNSLLVSWQPPRARIT

SEQ ID NO: 22 - protein, 22 aa, Artificial Sequence
  Description of Artificial Sequence Synthesized peptide
CFESEFGKIYGPFCECDNFSCA

SEQ ID NO: 23 - protein, 22 aa, Artificial Sequence
  Description of Artificial Sequence Synthesized peptide
CHLSPYGNIYGPYCQCDNFSCV

SEQ ID NO: 24 - protein, 22 aa, Artificial Sequence
  Description of Artificial Sequence Synthesized peptide
CHSSDFGKIYGKYCECDDFSCV

SEQ ID NO: 25 - protein, 23 aa, Artificial Sequence
  Description of Artificial Sequence Synthesized peptide
HTSDVPGKLIYGQYCECDTINCE

SEQ ID NO: 26 - protein, 24 aa, Artificial Sequence
  Description of Artificial Sequence Synthesized peptide
CRKRDNTNEIYSGKFCECDNFNCD

SEQ ID NO: 27 - protein, 18 aa, Artificial Sequence
  Description of Artificial Sequence Synthesized peptide
ADGACGYGRFSPPCGAAG

SEQ ID NO: 28 - protein, 9 aa, Artificial Sequence
  Description of Artificial Sequence Synthesized peptide
CPEELWWLC

SEQ ID NO: 29 - protein, 18 aa, Artificial Sequence
  Description of Artificial Sequence Synthesized peptide
ADIMINFGRWEHGDGYPF

SEQ ID NO: 30 - DNA, 30 bp, Artificial Sequence
  Description of Artificial Sequence Synthesized nucleotide
ggcggccatatgggaaacgcagatggcgcg

SEQ ID NO: 31 - DNA, 30 bp, Artificial Sequence
  Description of Artificial Sequence Synthesized nucleotide
ggctgcagttatccttggtcggggcagaag

SEQ ID NO: 32 - DNA, 30 bp, Artificial Sequence
  Description of Artificial Sequence Synthesized nucleotide
ggcggccatatggcccccagacagcgccag

SEQ ID NO: 33 - DNA, 31 bp, Artificial Sequence
  Description of Artificial Sequence Synthesized nucleotide
ggctgcagtcaaccatagaggtgccggatgc

SEQ ID NO: 34 - DNA, 28 bp, Artificial Sequence
  Description of Artificial Sequence Synthesized nucleotide
ggtggtctcgaggagtgccaggatgggg

SEQ ID NO: 35 - DNA, 36 bp, Artificial Sequence
  Description of Artificial Sequence Synthesized nucleotide
ggtggtgcggccgcttaagcgttacagttgtccccg

SEQ ID NO: 36 - protein, 18 aa, Artificial Sequence
  Description of Artificial Sequence Synthesiz ed peptide
ADGACRVYGPYLLCGAAG

SEQ ID NO: 37 - protein, 14 aa, Artificial Sequence
  Description of Artificial Sequence Synthesized peptide
ACGYTYHPPCARLT

SEQ ID NO: 38 - protein, 18 aa, Artificial Sequence
  Description of Artificial Sequence Synthesized peptide
ADGACPSYGPRFGCGAAG

SEQ ID NO: 39 - protein, 6 aa, Artificial Sequence
  Description of Artificial Sequence Synthesized peptide
GRGDSP

SEQ ID NO: 40 - protein, 17 aa, Artificial Sequence
  Description of Artificial Sequence Synthesized peptide
TPNSLLVSWQPPRARIT

SEQ ID NO: 41 - protein, 17 aa, Artificial Sequence
  Description of Artificial Sequence Synthesized peptide
PETLHPGRPQPPAEEEL

(19) United States
(12) Patent Application Publication
(10) Pub. No.: US 2007/0207967 A1
(43) Pub. Date: Sep. 6, 2007

(54) Peptide Inhibitors of Matrix Metalloproteinase Activity

(75) Inventors: Mikael Bjorklund, Helsinki (FI); Erkki Koivunen, Helsinki (FI)

Correspondence Address: BIRCH STEWART KOLASCH & BIRCH, PO BOX 747, FALLS CHURCH, VA, 22040-0747, US

(73) Assignee: CTT CANCER TARGETING TECHNOLOGIES OY, Helsinki (FI)

(21) Appl. No.: 11/578,793

(22) PCT Filed: Apr. 21, 2005
(86) PCT No.: PCT/FI05/50130

§ 371 Date: Feb. 1, 2007

(30) Foreign Application Priority Data
Apr. 23, 2004 (FI) 20040572

(51) Int. Cl. A61K 38/10; C07K 7/08

(52) U.S. Cl. 514/15; 530/328

(57) ABSTRACT

The present invention relates to novel matrix metalloproteinase (MMP) inhibitors and down-regulators, to pharmaceutical compositions comprising these inhibitors/down-regulators, to the improvement of liposome targeting to cancer cells, to the use of the novel MMP inhibitors for the manufacture of pharmaceutical and research preparations, to a method for inhibiting and down-regulating MMP-dependent conditions either in vivo or in vitro, to a method for inhibiting activations and/or functions as well catalytic and non-catalytic actions of matrix metalloproteinases, and to the use of the novel MMP inhibitors and down-regulators in biochemical isolation and purification procedures of matrix metalloproteinases.

### FIELD OF THE INVENTION

The present invention relates to novel inhibitors of matrix metalloproteinase (MMP) activity, to pharmaceutical compositions comprising these inhibitors, to the use of the novel matrix metalloproteinase inhibitors for the manufacture of pharmaceutical and research preparations, to a method for inhibiting and down-regulating MMP-dependent conditions either in vivo or in vitro, to a method for inhibiting catalytic and non-catalytic activities of matrix metalloproteinases, and to the use of the novel MMP inhibitors in biochemical isolation and purification procedures of matrix metalloproteinases.

### BACKGROUND OF THE INVENTION

Matrix metalloproteinases (MMPs) constitute a superfamily of genetically closely related proteolytic enzymes capable of degrading almost all the constituents of extracellular matrix and basement membrane that restrict cell movement. MMPs also process serpins, cytokines and growth factors as well as certain cell surface components. MMPs are thought to have a key role in mediating tissue remodeling and cell migration during morphogenesis and physiological situations such as wound healing, trophoblast implantation and endometrial menstrual breakdown. MMPs are further involved in processing and modification of molecular phenomena such as tissue remodeling, angiogenesis, cytokine, growth factor, integrin and their receptor processing. MMPs also mediate release and membrane-bound proteolytic processing of tumor necrosis factor (TNF-α) by bacterial-virulence factor induced monocytes. This event is mediated by a membrane-bound metalloproteinase TACE (TNF-α activating enzyme). Thus MMP-inhibitors, such as the novel peptides presented in this invention, can i.a. prevent activation of TNF-α by blocking this type of activating enzymes (see e.g. U.S. Pat. No. 6,624,144 (Koivunen et al)).

Matrix metalloproteinases (MMP)-2 and -9, also known as gelatinases play an important role in cell migration and tissue remodelling during development but also in pathological conditions such as inflammation and cancer (1). We have identified a highly selective peptide inhibitor of gelatinases, CTTHWGFTLC (CTT) by phage display (2) whereas others have developed gelatinase-selective small molecule inhibitors (3) to specifically target these enzymes.

The unique structural feature of the gelatinases is the collagen-binding domain (CBD) within the catalytic domain (4). The CBD is composed of three fibronectin type II repeats and is an intriguing target to develop gelatinase-specific compounds. Like most MMPs, the gelatinases also contain a C-terminal-hemopexin/vitronectin-like domain (C domain or PEX), which contains the binding site for tissue inhibitors for matrix metalloproteinases (TIMPs) and is responsible for the dimerization of MMP-9 (5).

Although MMP-2 and MMP-9 are closely related enzymes, they do have differences in the regulation of expression, activation, glycosylation and in substrate selectivity (1,4). Of these two enzymes MMP-2 has been investigated in a more detail. For example, the activation of pro-MMP-2 has been thoroughly characterized and involves interactions of TIMP-2, MT1-MMP and α_{V}β₃ integrin on the cell surface (6,7). MMP-9 has not been found to be activated via the same mechanism, and several proteinases including the plasmin/MMP-3 cascade (8) and trypsin-2 (9) can activate MMP-9 in vitro.

Relatively little is known about the molecular details of the MMP-9 interactions on the cell surface and how these regulate cell migration. MMP-9 has been found to interact with the α₅β₁ integrin, the α2 chain of type W collagen and the hyaluronan receptor CD44 (10,11). We have recently identified the leukocyte specific β₂-integrins as a binding partner for pro-MMP-9. The phage display peptide ADGACILWMDDGWCGAAG (DDGW) competed with pro-MMP-9 binding to the ligand-binding I domain of α_{M} integrin subunit and inhibited migration of leukocytes (12). Here we have isolated MMP-9 binding peptides, which inhibit either substrate binding or proenzyme activation leading to an inhibition of cell migration and invasion. Using these peptides, we identify MMP-9 interaction sites in fibronectin, vitronectin and αv₆₂ ₅ integrin.

Several studies have shown that the expression and activities of MMPs are pathologically elevated over the body's endogenous anti-proteinase shield in a variety of diseases such as cancer, metastatis, rheumatoid arthritis, multiple sclerosis, periodontitis, osteoporosis, osteosarcoma, osteomyelitis, bronchiectasis, chronic pulmonary obstructive disease, and skin and eye diseases. Proteolytic enzymes, especially MMPs, are believed to contribute to the tissue destruction damage associated with these diseases.

There is a variety of other disorders in which extracellular protein degradation/destruction plays a prominent role. Examples of such diseases include arthritides, acquired immune deficiency syndrome (AIDS), burns, wounds such as bed sores and varicose ulcers, fractures, trauma, inflammation, gastric ulceration, skin diseases such as acne and psoriasis, lichenoid lesions, epidermolysis bollosa, aftae (reactive oral ulcer), dental diseases such as periodontal diseases, peri-implantitis, jaw and other cysts and root canal treatment or endodontic treatment, related diseases, external and intrinsic root resorption, caries etc.

Although some inhibitors for MMPs do exist and have been investigated (for more information see U.S. Pat. No. 6,624,144 (Koivunen et al)), the tests are still mostly at the experimentation stage and no clinically acceptable inhibitor for MMPs exists as a therapeutic or prophylactic drug for any of the pathological states and diseases potentially connected with MMPs. Moreover, adverse side effects which have been detected in MMP inhibitors include, for instance, toxicities (synthetic peptides), antimicrobial activities (tetracyclines), etc. Thus, there is a continuous need in the field for novel therapeutically promising candidate compounds for MMP-inhibition.

### BRIEF DESCRIPTION OF THE DRAWINGS

FIG. 1. Characterization of peptide ligands to MMP-9. (A) Phage carrying PPC, CRV or a control peptide (CPEELWWLC) were allowed to bind to pro-MMP-9 with the indicated peptides (20 μM) or gelatin (2.5 μg/ml) as a competitor. Bound phage was quantified with an anti-phage antibody. The results are mean±SD of triplicate samples in this and other figures unless otherwise stated. **, statistically significant difference (p<0.001) in Student's t test in this and other figures. (B) PPC, but not CTT or CRV, inhibits binding of gelatin to immobilized CBD. Biotinylated gelatin was detected using a streptavidin-peroxidase conjugate. (C) The enzymatic activity of MMP-2 and MMP-9 in a gelatin degradation assay is inhibited by the PPC peptide but not the CRV peptide or the scrambled PPC peptide. (D) Binding of the biotinylated CBD to intact fibronectin, the 110-kDa cell-binding fragment of fibronectin, vitronectin (1 μg/well) or BSA in the presence or absence of 20 μM peptides was measured. (1D, insert) Binding of the biotinylated CBD to fibronectin-derived peptide TTPNSLLVSWQPPRARIT or a control peptide on a pepspot filter. CBD binding was detected with enhanced chemiluminescence.

FIG. 2. CRV peptide selectively binds to the C-terminal domain of MMP-9 and inhibits homodimerization. (A) Binding of the CRV phage to pro-MMPs (A) and the recombinant C terminal domains of MMP-2 and -9 (B) was studied in the presence or absence of 20 μM soluble peptides. (C) Phage binding to the recombinant C domain or MMP-9 or the CBD was assayed in the presence or absence of peptides (20 μM) or TIMP-1 (2.5 μg/ml). (D) Binding of the ¹²⁵I-labelled C domain to unlabelled C domain, CBD or BSA was studied in the presence or absence of CRV or scrambled CRV peptide. Bound radioactivity was measured with a gamma-counter. **, statistically significant difference (p<0.001) in Student's t test. n.s., not significant.

FIG. 3. MMP-9 domain-specific inhibition of cell migration and invasion. (A) HT1080 fibrosarcoma invasion through matrigel-coated invasion chambers in the presence or absence of the peptides. All samples were assayed in triplicates in three independent experiments. (B) Transwells were coated with LLG-C4-GST or GST as a control. THP-1 cells were allowed to migrate for overnight in the presence of peptides. (C) Gelatinolysis of HT1080 cells after a 48 hour incubation with the peptides in the presence or absence of 20 nM PDBu. Data is mean±SEM from six samples. (D) Pro-MMP-9 binding to the α_{M} integrin I domain in the presence or absence of peptides. Bound MMP-9 was detected with a monoclonal anti-MMP-9 antibody. Statistically significant differences in t test are indicated with asterisks *, p<0.05 and **, p<0.001. (E) Binding of gelatin to pro-MMP-9/integrin complex. Biotinylated gelatin was detected with streptavidin peroxidase. Activation of MMP-9 in HT1080 (F) and THP-1 (G) cells. The cells were incubated in serum-free medium in the presence of phorbol ester to stimulate MMP-9 expression. Plasminogen (2.5 μg/ml) and pro-MMP-3 (0.5 μg/ml) were added to promote MMP-9 activation. The peptides were used at a 200 μM concentration or as indicated. The samples were analyzed by gelatin zymography. (H) Activation of pro-MMP-9 by MMP-3 in vitro in the presence of CRV and scrambled peptide (200 μM).

FIG. 4. MMP-9 interacts with and cleaves uPAR. (A) Immunoprecipitations with antibodies to uPAR (399R) and MMP-9 (H-129) were performed from BDBu-activated HT1080 cells and non-activated and activated THP-1 cells. Pro-MMP-9 was detected with western blotting. (B) MMP-9 cleaves soluble uPAR in vitro. The cleavage is inhibited with 10 mM EDTA. Chymotrypsin cleavage, which yields a D2D3 fragment is shown as a control. (C) Inhibition of uPAR cleavage on HT1080 by a chemical gelatinase inhibitor InhI (20 μM). DMSO was used as vehicle for the InhI. Aprotinin (25 μg/ml) and benzamidine (20 μM) were used as controls. Equal amounts of membrane fractions were separated on SDS-PAGE and analyzed with antibodies to uPAR. The conditioned medium was analyzed by gelatin zymography. The cell surface gelatinases and uPA were analyzed from acid eluates. (D) uPAR cleavage on THP-1 cells is similarly inhibited by the gelatinase inhibitors InhI (20 μM) and CTT (200 μM).

FIG. 5. Identification of the integrin β₅ chain as a binding site for MMP-9 C domain. (A) Rabbit antisera against the cytoplasmic domain of integrins were used for immunoprecipitation followed by western blotting with anti-MMP-9 antibodies as in FIG. 4. (B) Schematic representation of the integrin β chain. The sequence similar to the CRV peptide in individual β chains is shown. The KIM127 antibody epitope in the β₂ integrin is underlined. (C) The CRV peptide or the MMP-9 domains do not block HT1080 cell adhesion to fibronectin or vitronectin. (D) Binding of biotinylated I-EGF2+3 fragment of β₅ integrin to the C domain or CBD of MMP-9, BSA, I-EGF2+3 fragment or vitronectin was assessed in the presence or absence of peptides or unlabelled EGF2+3. ** indicates p<0.001 in Student's t test (E) Competition of β₅ I-EGF2+3 fragment binding by the alanine mutants of β₅ EGF2+3. (F) Inhibition of HT1080 invasion through matrigel in the presence or absence of β₅ integrin and MMP domains (50 μg/ml). The data is mean±SD from four samples. Statistically significant differences in t test are indicated with asterisks *, p<0.05 and **, p<0.001.

FIG. 6. Cell surface interactions of MMP-9 C domain and integrin β chain. (A) Binding of ¹²⁵I-labelled MMP-9 C domain to the wild type and β₅ integrin-transfected CS-1 cells in the presence or absence of unlabelled proteins (50 μg/ml) or peptides (50 μM) as competitors. The data represents mean±SEM from 5-8 datapoints. (B) Inhibition of THP-1 cell binding to immobilized KIM127 was assayed in the presence or absence of MMP-9 domains or antibodies. Statistical differences were determined with the t test.

FIG. 7. MMP-9, uPAR and integrins β₅ integrins colocalize in the leading edge of HT1080 cells. The cells adhered on vitronectin coated coverslips were incubated overnight in a serum gradient and in the presence of PDBu to stimulate migration. The cells were stained with the respective antibodies and observed under fluorescence microscope. Bar 25 μM.

FIG. 8. CRV peptide inhibits the growth of HSC-3 human tumor xenografts in vivo. (A) Tumor volumes of CRV (ten tumors), scrambled CRV (eight tumors) or PBS (ten tumors) injected mice measured at day 31 or before at the time the tumor size reached the end-point of 1000 mm³ (broken line). Bars represent mean tumor volumes from each experimental condition. Statistical significance was calculated with the t test. (B) Kaplan-Meier survival analysis of CRV and PBS (five mice per group), and scrambled CRV (four mice per group). (CRV vs. scrambled CRV; Log-Rank test p=0.003, CRV vs. PBS; p=0.002, PBS vs. scrambled CRV; p=0.11) (C) Tumor vasculature was stained with an anti-CD31 antibody. Representative samples from the CRV, scr. CRV and PBS treated mice. Bar 200 μM.

### DETAILED DESCRIPTION OF THE INVENTION

The abbreviations used herein are: MMP, matrix metalloproteinase; CBD, collagen binding domain; C domain, C-terminal hemopexin-like domain; pro-MMP-9-ΔHC, pro-MMP-9 lacking the hinge region and the C domain; TIMP, tissue inhibitor of MMPs; uPA, urokinase-plasminogen activator; uPAR, uPA receptor; CTT, CTTHWGFTLC peptide; CRV, CRVYGPYLLC peptide; PPC, ADGACGYGRFSPPCGAAG peptide; DDGW, ADGACILWMDDGWCGAAG peptide, PDBu, phorbol ester; VN, vitronectin; FN, fibronectin.

Migration of invasive cells appears to be dependent on matrix metalloproteinases (MMPs) anchored on the cell surface through integrins. We have previously demonstrated an interaction between the integrin α-subunit I domain and the catalytic domain of MMP-9. We now show that there is also an interaction between the integrin β subunit and MMP-9. Using phage display we have developed MMP-9 inhibitors that bind either to the MMP-9 catalytic domain, collagen binding domain or the C-terminal hemopexin-like domain. The C-terminal domain-binding peptide mimicks an activation epitope in the stalk of the integrin β chain, and inhibits the association of MMP-9 C-terminal domain with αVβ₅ integrin. Unlike other MMP-9 binding peptides, it does not directly inhibit catalytic activity of MMP-9, but still prevents proenzyme activation and cell migration in vitro, and tumor xenograft growth in vivo. We also find an association between MMP-9 and urokinase-plasminogen activator receptor (uPAR), and that uPAR is cleaved by MMP-9. Collectively, we have defined molecular details for several interactions mediated by the different MMP-9 domains.

It is therefore an object of the present invention to provide novel matrix metalloproteinase inhibitors and binding-ligands based on the structure of the peptide motif
CG(Ar)GR(Ar)(S/Q)PPC
which corresponds to the sequences shown in SEQ ID NO:1 and SEQ ID NO:2 of the sequence listing and wherein Ar is any aromatic amino acid residue (i.e. Phe, Trp, or Tyr), or on the structure of the peptide motif
CRXYGPXXXC
which corresponds to the sequence shown in SEQ ID No. 3, wherein X is any amino acid residue.

The present invention also relates to a pharmaceutical composition comprising an amount of the novel matrix metalloproteinase inhibitor(s)/down-regulator(s) effective to reduce the activities, activations, functions, and/or expressions of one or more MMPs, especially of MMP-2 and/or MMP-9, and a pharmaceutically and biochemically acceptable carrier. Pharmaceutical compositions comprising novel MMP inhibitor(s)/downregulator(s) according to the invention may be used systemically, locally and/or topically. They also include all potential combinations (combo-medications) with other MMP-inhibitors, other drugs and tumor-homing chemicals/molecules.

The present invention also includes the use of the novel matrix metalloproteinase inhibitors for the manufacture of pharmaceutical preparations for the treatment of matrix metalloproteinase dependent conditions, and also their use, for example as affinity ligands, in biochemical purification and isolation procedures of MMPs. The MMP-dependent conditions include, but are not limited to, wounds, burns, fractures, lesions, inflammations, ulcers, cancer and metastasis progression in connective tissues and bone, periodontitis, gingivitis, peri-implantitis, cysts, root canal treatment, internal and external root canal resorption, caries, AIDS, corneal ulceration, gastric ulceration, aftae, trauma, acne, psoriasis, loosening of the end-osseal hip-prosthesis, osteomyelitis, osteoporosis, tissue remodeling, angiogenesis, arthritides (rheumatoid, reactive and osteo arthritides), angiogenesis, lung diseases (bronchiectasis and chronic obstructive pulmonary diseases and other lung diseases).

The present invention also relates to a process for the preparation of novel matrix metalloproteinases which process comprises standard solid-phase Merrifield peptide synthesis.

Especially preferred MMP inhibitors according to the present invention are peptide inhibitors CGYGRFSPPC and CRVYGPYLLC, which inhibit the activity of pro-MMP-9 as shown in the Experimental Section.

The novel peptide inhibitors we have developed are useful lead compounds to design peptidomimetics to block MMPs and cell migration. The above motifs may also be utilized to develop more selective inhibitors to individual members of the MMP family. Finally, the small size of the MMP-targeting peptides can be utilized to carry drugs to tumors. Phage-library derived peptides targeting receptors in tumor vasculature have been found to be useful cytotoxic drug carriers to tumors in mice. MMPs are potential receptors for targeted chemotherapy, because they are usually overexpressed in tumors as compared to normal tissues and appear to be involved in the angiogenic process.

Consequently, the invention is directed to the use of peptide compounds having the motif of CG(Ar)GR(Ar)(S/Q)PPC, wherein Ar is any aromatic amino acid, or peptide compounds having the motif of CRXYGPXXXC, wherein X is any amino acid residue, in improving targeting of liposomes to tumor cells, or in enhancing the uptake of liposomes to tumor cells (see WO02076491 for more details).

Thus, as a result of the invention, MMP dependent conditions may now be treated or prevented either with the novel MMP inhibitors alone or in combination with other drugs normally used in connection with the disease or disorder in question. These include for example tetracyclines, chemically modified tetracyclines (Golub et al., 1992), bisphosphonates, as well as homing/carrier molecules to the sites of tumors, such as integrin-binding peptides (Arap et al., 1998). The amount of novel matrix metalloproteinase inhibitors to be used in the pharmaceutical compositions according to the present invention varies depending on the specific inhibitor used, the patient and disease to be treated as well as the route of administration.

The novel MMP inhibitors of the present invention have shown no toxicity when injected into animals and do not affect cell number or viability.

The present invention thus also relates to a method for the therapeutic or prophylactic treatment of MMP-dependent conditions in mammals by administering to said mammal an effective amount of the novel MMP-inhibitor(s), as well as to a method for inhibiting the formations, synthesis, expressions, activations, functions and actions of MMPs in mammals by administering the novel MMP-inhibitor(s)/down-regulator(s) in an amount which is effective in blocking the formation, activation and actions of MMPs.

Furthermore, the present invention also relates to a method for the therapeutic or prophylactic treatment of THP-1-dependent conditions, such as inflammations, in mammals by administering to said mammal an effective amount of the novel MMP-inhibitor(s) of the invention, as well as to a method for inhibiting the activations, functions and actions of THP-1 cells in mammals by administering the novel MMP-inhibitor(s)/down-regulator(s) in an amount which is effective in blocking activation and actions of THP-1 cells.

The present invention also relates to a method for inhibiting matrix metalloproteinases in vitro comprising adding to an in vitro system the novel matrix metalloproteinase inhibitor(s) in an amount which is effective in inhibiting the MMP activity.

A further object of the invention is a method for isolating and purifying matrix metalloproteinases with the aid of the novel matrix metalloproteinase inhibitor(s).

The publications and other materials used herein to illuminate the background of the invention, and in particular, to provide additional details with respect to its practice, are incorporated herein by reference. The invention will be described in more detail in the following Experimental Section.

Experimental Section

Phage display. Phage display selections were made using random peptide libraries CX₇₋₁₀C and X₉₋₁₀ (13). Purified human pro-MMP-9 (9) or recombinant MMP-9 C domain (2 μg/ml) was immobilized on microtiter wells and the wells were blocked with BSA. The phage were added in 50 mM Hepes (pH 7.5)/5 mM CaCl₂/1 μM ZnCl₂/150 mM NaCl/2% BSA. After three rounds of selection the phage sequences were determined (14). The phage binding specificity was tested with pro-MMPs or the recombinant domains (20 ng/well). The phage (10⁸ transducing units/well) were allowed to bind in the absence or presence of competitor peptides (20 μM), gelatin (2.5 μg/ml) or TIMP-1 (2.5 μg/ml, Calbiochem) followed by washings with PBS/0.05% Tween20 (PBST). The phage were detected with a peroxidase-conjugated anti-phage antibody (Amersham Biosciences).

Peptide synthesis. The phage peptides were initially prepared in a recombinant form using intein fusions (12,15). Chemical peptide synthesis was done using Fmoc-chemistry and the purity and integrity of the peptides was verified by mass spectroscopy (15). The peptides were dissolved in water, except the CRV and DDGW peptides, which were dissolved in 50 mM NaOH at a 10 mM concentration and then diluted into PBS to neutralize the pH. The TTPNSLLVSWQPPRARIT and ADIMINFGRWEHGDGYPF peptides were synthesized on a cellulose membrane. The membrane was blocked with 3% BSA in TBS/0.05% Tween 20, and incubated with 0.2 μg/ml biotinylated CBD. Bound CBD was detected using peroxidase-conjugated streptavidin (1:10 000 dilution, Pierce) and chemiluminescence detection.

Expression of the MMP and integrin domains. CBD (amino acids Gly²⁰⁴-Gly³⁷³) was amplified from MMP-9 cDNA with the oligonucleotides 5′-GGCGGCCATATGGGAAACGCAGATGGCGCG-3′ and 5′-GGCTGCAGTTATCCTTGGTCGGGGCAGAAG-3′ incorporating NdeI and PstI restriction sites. The PCR product was ligated into pTWIN vector (New England Biolabs). CBD was expressed in E. coli and purified using gelatin-sepharose (Amersham Biosciences). For some experiments, CBD was biotinylated with sulfo-NHS-LC-biotin (Pierce). The C-terminal domains of MMP-2 (Glu⁴³⁸-Cys⁶³¹) and MMP-9 (Asp⁴⁹⁴-Asp⁶⁸⁸) were expressed as described (16). The pro-MMP-9-ΔHC (Ala¹-Gly⁴²⁴) was cloned with the oligonucleotides 5′-GGCGGCCATATGGCCCCCAGACAGCGCCAG-3′ and 5′-GGCTGCAGTCAACCATAGAGGTGCCGGATGC-3′, digested with NdeI and PstI and ligated into the pTWIN vector. The protein was purified from inclusion bodies by solubilization with urea, refolded in the presence of arginine and purified with gelatin-sepharose. The integrin β₅ I-EGF2+3 fragment (Glu⁴⁷⁶-Asn⁵⁶³) was cloned from β₅ integrin cDNA using oligonucleotides 5′-GGTGGTCTCGAGGAGTGCCAGGATGGGG-3′ and 5′-GGTGGTGCGGCCGCTTAAGCGTTACAGTTGTCCCCG-3′, digested with XhoI and NotI and ligated into pHAT2 vector. The protein with an N-terminal His6-tag was expressed in E. coli and purified in a soluble form using Ni²⁺-affinity chromatography. The K542A and Y544A mutant β₅ I-EGF2+3 constructs were prepared by site directed mutagenesis. The integrity of all constructs was verified by DNA sequencing.

Gelatinase inhibition assay. Inhibition of aminophenylmercuric acetate (APMA)-activated MMP-2 and trypsin-activated MMP-9 was performed using biotinylated gelatin as a substrate (15).

Gelatin and CBD binding assays. Recombinant CBD or human plasma fibronectin (Calbiochem) (0.2 μg/ml in TBS) was immobilized in microtiter wells. The wells were saturated with 1% BSA-PBST. Biotinylated gelatin (0.2 μg/ml in 1% BSA-PBST) was added with or without peptides at the concentrations indicated or with an excess of unlabelled gelatin (10 μg/ml) and allowed to bind for 1 h. Bound gelatin was detected with streptavidin-peroxidase. CBD binding to immobilized fibronectin, the 110-kDa fragment of fibronectin (Upstate Biotechnology) or urea-denaturated human plasma vitronectin (17) (1 μg/well) was studied using biotinylated CBD (5 μg/ml) in 1% BSA-PBST in the presence or absence of 20 μM peptides.

Dimerization of the MMP-9 C domain. Recombinant C domain or CBD at a 5 μg/ml concentration in PBS were coated on microtiter wells followed by blocking with 1% BSA-PBST. ¹²⁵I-labelled C domain was preincubated with the peptides for 30 minutes in 1% BSA-PBST and then added to the wells. After a 2 hour incubation, the wells were washed. Bound radioactivity was eluted with 1% SDS and measured with a gamma-counter.

Cell culture. Human HT1080 fibrosarcoma, monocytic THP-1 and HSC-3 tongue squamous cell carcinoma cells were maintained as described (2,14,18). Wild type and β₅ integrin-transfected CS-1 hamster melanoma cells were kindly provided by Dr. David Cheresh from the Scripps Research Institute and maintained as described (19). Cell viability was measured using an MTT (3-[4,5-dimethylthiazol-2-yl]-2,5-diphenyl tetrazolium bromide) assay (Roche).

Cell adhesion. HT1080 cells were allowed to adhere on vitronectin or fibronectin (2 μg/ml) in the presence or absence of peptides (200 μM), proteins (40 μg/ml) or a monoclonal anti-αvβ₅ integrin antibody P1F6 (Chemicon) or a control antibody (25 μg/ml). The adhesion was quantified as described (14). Adhesion of THP-1 cells to immobilized KIM127 or control monoclonal antibodies (2 μg/ml) was done in the presence or absence of soluble proteins (50 μg/ml) or antibodies (25 μg/ml).

Cell migration and invasion. The cell migration assay was conducted using transwell migration chambers (8 μm pore size, Costar) in 10% serum-containing medium (2,14). Briefly, the membranes were coated on both sides with 40 μg/ml GST or with the β₂ integrin ligand peptide CPCFLLGCC-GST fusion (GST-LLG-C4) and blocked with complete medium. THP-1 cells (50 000/100 μl) were preincubated with the peptides for 1 h in serum-containing medium. The cells were allowed to migrate for 16 h and were then stained with crystal violet and counted (14). The HT1080 (20 000 cells/I100 μl) invasion assay was performed as the THP-1 migration, except that matrigel coated transwells (BD Biosciences) were used.

Pericellular proteolysis. Microtiter wells were coated with a mixture of fibronectin (10 μg/ml) and FITC-labelled gelatin (100 μg/ml) followed by saturation with 1% BSA in PBS. HT1080 cells (50 000 in 100 μl 0.1% BSA/DMEM) were incubated in the presence of 20 nM PDBu (4β-phorbol-12,13-dibutyrate, SigmaAldrich) and the peptides or the MMP-2/MMP-9 selective inhibitor InhI (Calbiochem). As a control non-activated cells and medium without the cells were used. Gelatinolysis after 48 hours was measured as the increase of fluorescence from a 50 μl aliquot of the conditioned medium using Wallac Victor² reader.

Pro-MMP-9 and gelatin binding to leukocyte α_{M} integrin. Pro-MMP-9 binding to the α_{M} I domain in the presence of peptides was studied as described (12). Gelatin binding to the pro-MMP-⁹/α_{M}β₂ integrin complex was studied by immobilizing the integrin α_{M}β₂ (12) or α_{IIb}β₃ as a control (Enzyme Research Laboratories, South Bend, Ind.) (1 μg/well) in TBS/1 mM CaCl₂/1 mM MgCl₂ followed by saturation of the wells with 1% BSA in PB ST. Pro-MMP-9 (100 ng/well) was incubated for 2 h and the unbound pro-MMP-9 was washed away. Biotinylated gelatin (2.5 μg/ml) was allowed to bind for 30 min at room temperature. Bound gelatin was detected with streptavidin-peroxidase.

Activation of MMP-9. THP-1 cells (40 000/100 μl) or confluent HT1080 cells were incubated for 16 h in the presence or absence of 2.5 μg/ml plasminogen, 0.5 μg/ml pro-MMP-3 (Oncogene Research Products), 40 nM PDBu, and the peptides at a 200 μM concentration unless otherwise indicated. Aliquots of the conditioned media were analyzed by gelatin zymography (9). MMP-9 was activated in vitro with MMP-3 (1:5 enzyme to substrate) in 50 mM Tris-HCl (pH 7.5)/5 mM CaCl₂,/1 μM ZnCl₂/0.02% NaN₃/0.01% Tween20 for 1 h at +37° C. in the presence or absence of 200 μM CRV or the scrambled peptide. The samples were analyzed by gelatin zymography.

Immunoprecipitation and western blotting. HT1080 cells were activated with 50 nM PDBu for 3 h in serum-free medium, washed with PBS and lysed in 10 mM Tris-HCl (pH 8.0)/140 mM NaCl/1% Triton X-100/1 mM PMSF. One milligram of protein was immunoprecipitated with 4 μg of anti-uPAR (399R, American Diagnostica, Greenwich, Conn.) or anti-MMP-9 (H-129, SantaCruz Biotechnology) or a control IgG. Integrins were immunoprecipitated with 2 μl of anti-integrin cytoplasmic domain antisera (20). The immunoprecipitates were resolved on an 8% SDS-PAGE gel, blotted and detected with anti-MMP-9 antibodies.

Immunofluorescence. HT1080 cells were allowed to adhere on vitronectin (10 μg/ml) in serum-free DMEM. Directional migration of the cells was stimulated by overlaying the cells with 0.5% agarose in DMEM and adding 5 μl FBS with PDBu (20 nM final concentration) to the one end of the wells. Overnight cultured cells were washed with PBS, fixed with paraformaldehyde, permeabilized, and stained with the monoclonal anti-uPAR antibody (Ab3937, American Diagnostica, 2 μg/ml) or anti-β5 integrin IA9 (2 μg/ml, (21)) and polyclonal MMP-9 antibodies (H-129, 10 μg/ml). The primary antibodies were detected with anti-mouse Alexa Fluor 488 and anti-rabbit Alexa Fluor 555 antibodies.

uPAR clevage. 0.5 μg recombinant soluble human uPAR (suPAR, R&D Systems) was digested with 50 ng trypsin-activated MMP-9 in 50 mM Tris-HCl (pH 7.5)/5 mM CaCl₂/1 μM ZnCl₂/0.02% NaN₃/10 μg/ml aprotinin with or without 10 mM EDTA. Chymotrypsin cleavage was done without aprotinin. The samples were incubated for 16 hours in 37° C. and separated in a non-reducing 12% SDS-PAGE followed by western blotting with anti-uPAR antibodies (399R, 1:1000 dilution). uPAR cleavage on the surface of HT1080 cells or THP-1 cells was studied in a serum-free medium with or without 20 nM PDBu for 48 h in the presence or absence of 20 μM InhI, 200 μM CTT or W→Λ CTT control peptide, 25 μg/ml aprotinin or 20 μM benzamidine. The cells were washed three times with PBS, incubated with 50 mM glycine-HCl (pH 3.0)/100 mM NaCl to extract cell surface bound urokinase-plasminogen activator (uPA) and MMPs, and neutralized with 500 mM Hepes (pH 7.5)/100 mM NaCl. Membrane proteins were enriched by Triton X-114 extraction (22) and 30 μg (HT1080 cells) or 10 μg (THP-1 cells) protein was separated on 12% SDS-PAGE and analyzed for uPAR as above. Gelatinases were analyzed from the acid eluates with gelatin zymography and uPA with plasminogen/milk-powder zymography (23).

B5 I-EGF2+3 binding assay. MMP-9 C domain, MMP-2 C domain, CBD, β₅ I-EGF2+3 or vitronectin (2 μg/ml) were immobilized in microtiter wells. Biotinylated β5 I-EGF2+3 fragment (2.5 μg/ml) was added to the wells which were preincubated with the competitors for 30 min in 1% BSA-PBST and then incubated further for one hour. Bound biotinylated protein was detected with streptavidin-peroxidase.

¹²⁵I-C domain binding to cells. The MMP-9 C domain was labelled with ¹²⁵I to a specific activity 0.06 μCi/pmol. The labelled domain retained 40% of the CRV peptide-binding activity as shown by the phage-binding assay. The CS-1 cells were washed with 2.5 mM EDTA in PBS and suspended in 20 mM Hepes (pH 7.5)/150 mM NaC1/1 mM MnCl₂/0.2 mM CaCl₂/0.5% BSA. After a preincubation of 1.5×10⁶ cells in a 200 μl volume on ice for 30 min with the competitors, ¹²⁵I-labelled C domain (1×10⁶ cpm) was added and incubated for three hours on ice. The cells were transferred to tubes containing 200 μl of dibutyl phthalate/cyclohexane mixture (23:2 vol/vol), centrifuged 7500×g for 10 minutes and snap-frozen (24). The bottom of the tube containing the cells were cut and analyzed with a gamma-counter.

Tumor growth in vivo. The animal studies were approved by the ethical committee of Helsinki University. HSC-3 tumors were established by administering 5×10⁶ tumor cells in a 100 μl volume in PBS in both flanks of the Hsd:Athymic Nude-nu mice. After three days, the mice received five daily injection of 0.8 mg/ml CRV or the scrambled peptide or the vehicle (PBS) via the tail vein in a 200 μl volume. Three-dimensional caliper measurements were taken twice a week and the tumor volumes calculated. Mice were sacrificed when the tumor volume reached 1000 mm³. For the staining of the tumor vasculature, 7 μm frozen tissue sections were stained with anti-CD31 antibody (MEC 13.3, BD Biosciences) and anti-rat Alexa Fluor 488 antibodies.

Statistical analysis. Statistical significance was calculated with the t test or with Log-Rank test in Kaplan-Meier survival analysis.

Results

Identification of peptide probes to different domains of MMP-9—In order to understand gelatinase-mediated cell migration in depth, we searched for putative MMP-9-binding proteins by phage display of random peptide libraries. The pro-MMP-9 and its recombinant domains were used in biopanning as the active MMP-9 primarily bound peptides with a WGF motif (2). Two groups of pro-MMP-9 binding peptides were found (Table 1). The group I had a motif CG(Ar)GR(Ar)(S/Q)PPC, where Ar is an aromatic amino acid. These peptides show similarity to sequences found in the gelatinase substrates fibronectin and vitronectin (4). The group II had a CRXYGPXXXC motif. In this group, the CRVYGPYLLC peptide was obtained by biopanning with pro-MMP-9, whereas the other sequences were obtained with a recombinant C-terminal domain. The CGYGRFSPPC (PPC) and CRVYGPYLLC (CRV) peptides were chosen for further studies as representatives of the two groups.

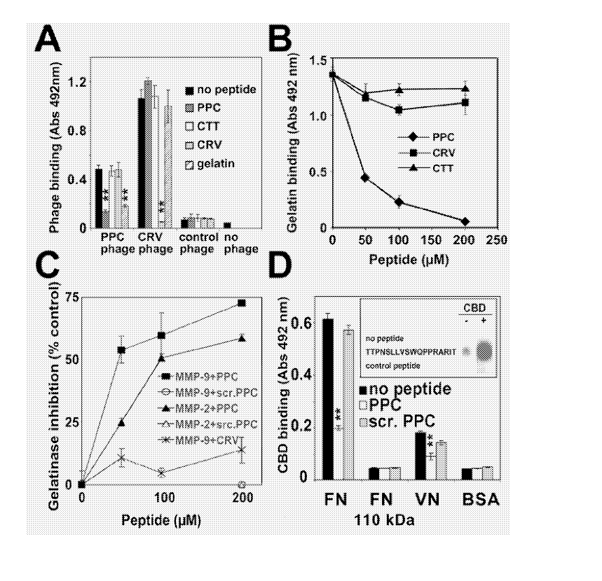

To identify the binding sites of these peptide motifs, we carried out phage binding experiments. Binding of PPC peptide-bearing phage to pro-MMP-9 was inhibited by a soluble recombinant 18-mer ADGACGYGRFSPPCGAAG (PPC) peptide and gelatin, but not with CTT or a recombinant ADGACRVYGPYLLCGAAG (CRV) peptide (FIG. 1A). Conversely, binding of the CRV-phage was inhibited by CRV and not by PPC, CTT or gelatin, indicating non-overlapping binding sites for these peptides. Inhibition of PPC phage binding by gelatin implied that that PPC binds to the collagen-binding domain (CBD) of MMP-9. Furthermore, phage selection with the MMP-2 CBD has also yielded a PPC-like peptide ACGYTYHPPCARLT (25). The PPC peptide, but not CTT or CRV, inhibited gelatin binding to immobilized CBD in a dose dependent manner (FIG. 1B), but had no effect on gelatin binding to fibronectin (data not shown) suggesting that PPC is specific for the fibronectin type II repeats of gelatinases. In a gelatin degradation assay, PPC inhibited both MMP-9 and MMP-2 activity (FIG. 1C), the scrambled control peptide ADGACPSYGPRFGCGAAG (scr. PPC) having no effect. The CRV peptide did not inhibit gelatinase activity consistent with the inability of gelatin to compete with CRV. The PPC peptide was a weaker gelatinase inhibitor than CTT, which completely inhibits gelatin degradation at a 100 μM concentration in this assay (15).

To study whether the PPC-like sequences of fibronectin and vitronectin bind MMP-9, we examined the binding of CBD to these proteins in a solid phase binding assay. CBD bound to both fibronectin and vitronectin, but not to the 110-kDa fragment of fibronectin lacking the C-terminal heparin-binding domain and thus the suspected gelatinase-binding site (FIG. 1D). PPC, but not the scrambled peptide inhibited the CBD binding. Similar results were obtained in a pepspot membrane assay, where biotinylated CBD bound to the PPC-like fibronectin peptide TTPNSLLVSWQPPRARIT but not to an 18-mer control peptide (FIG. 1D, insert).

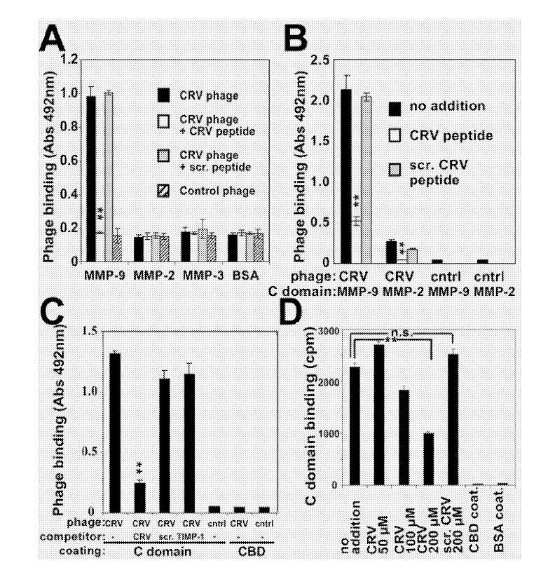

When different MMPs were compared, the CRV phage showed a peptide-inhibitable binding only to pro-MMP-9, and not to pro-MMP-2 or pro-MMP-3 (FIG. 2A). MMP-9 selectivity was also observed with the recombinant MMP-9 and MMP-2 C domains. The CRV phage recognized the MMP-9 C domain strongly in comparison to the MMP-2 C domain (FIG. 2B). TIMP-1 could not compete with the CRV phage binding to the MMP-9 C domain (FIG. 2C) or pro-MMP-9 (not shown). The CRV phage did not bind to the CBD (FIG. 2C) or a pro-MMP-9 lacking the hinge region and the C-terminal domain (pro-MMP-9-ΔHC, not shown). We next examined the effect of CRV on the dimerization of MMP-9 C domain. ¹²⁵I-labelled C domain was preincubated with CRV or scrambled peptide and then added to wells coated with unlabelled C domain. Dimerization of the C domain was inhibited by CRV, but not by the scrambled peptide (FIG. 2D).

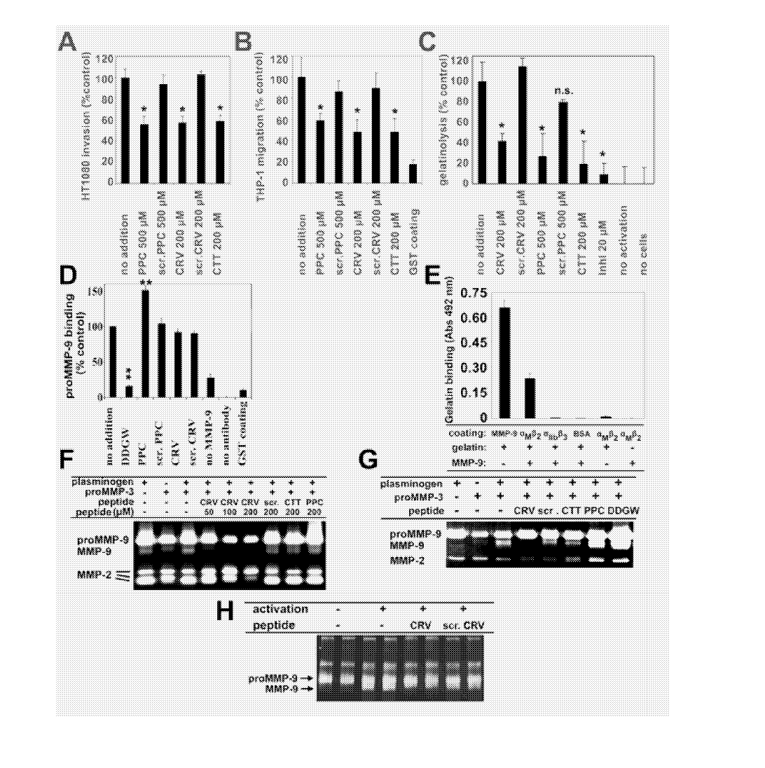

Cell migration and invasion are inhibited by blocking the domain-specific interactions of the gelatinases—We studied the role of the gelatinase domains in cell migration and invasion using the CTT, PPC and CRV peptides. The binding site of CTT maps to the catalytic domain, but not to CBD (FIG. 1B, (12) and our unpublished data). As indicated above, PPC and CRV are probes for the CBD and the C domain, respectively. All three peptides inhibited HT1080 fibrosarcoma invasion into matrigel. At a 200 μM concentration of CRV or CTT, 50% inhibition was observed. The PPC peptide required a 500 μM concentration to achieve the same efficacy (FIG. 3A). The scrambled control peptides were inactive. Similar results were obtained with THP-1 monocytic cells, which migrate on a synthetic GST-LLG-C4 substratum (14) in a β₂ integrin and gelatinase dependent manner. PPC, CRV and CTT, but not the scrambled peptides had an inhibitory effect (FIG. 3B). The inhibition of cell migration was not due to toxicity as there was no effect on cell viability when the cells were cultured for 48 hours with the peptides at a 500 μM concentration (not shown). Surprisingly, CRV inhibited pericellular gelatinolysis similarly as CTT and PPC did, as measured by a release of fluorescent gelatin fragments into the conditioned medium (FIG. 3C). In this assay, HT1080 cells were cultured for 48 h in the presence of phorbol ester (PDBu) on a fibronectin/FITC-labelled gelatin coating. The gelatinase-selective small molecule inhibitor (Inh1) also inhibited gelatinolysis, but the scrambled peptides did not. These results indicated that not only the direct MMP enzyme inhibitors but also CRV affects cell migration and pericellular proteolysis. We also tested that the CRV and PPC peptides do not affect the interaction of MMP-9 with the leukocyte α_{M} integrin I domain, which is blocked by DDGW (FIG. 3D). In fact, PPC stabilized pro-MMP-9 binding to the I domain as shown by typically 20-50% higher binding in the presence of PPC. Antibody binding to pro-MMP-9 in the absence of the I domain was not affected by PPC (not shown). The data suggested that the α_{M}β₂ integrin-bound MMP-9 could bind its substrates using CBD to generate a triple molecular complex between an integrin, MMP-9 and a ligand/substrate. To directly test this, pro-MMP-9 was allowed to bind to immobilized α_{M}β₂ integrin and binding of biotinylated gelatin, a MMP-9 substrate, was examined. Gelatin bound to the pro-MMP-⁹/α_{M}β₂ integrin complex, but not the α_{M}β₂ integrin alone. The platelet integrin α_{IIb}β₃ did not support proMMP-9/gelatin-binding (FIG. 3E).

MMP-9 associates with the urokinase-plasminogen activator receptor—We next investigated the effects of the peptides on plasmin/MMP-3-mediated pro-MMP-9 activation in PDBu-activated HT1080 and THP-1 cells. The conditioned medium from the cells incubated in the presence of the peptides was analyzed by gelatin zymography. Of the three peptides, only CRV was capable of inhibiting pro-MMP-9 activation. In HT1080 cells, CRV peptide inhibited pro-MMP-9 activation strongly and the activation of pro-MMP-2 partially (FIG. 3F). Addition of plasminogen was sufficient in activating pro-MMP-9 in HT1080 cells and pro-MMP-3 did not promote activation any further. In THP-1 cells, pro-MMP-9 activation required pro-MMP-3 and plasminogen added together and the activation was blocked by CRV but not by the other peptides (FIG. 3G). In fact, pro-MMP-9 activation was augmented in the presence of PPC or DDGW and there were higher levels of released MMP-9 as previously observed with DDGW (12). CRV did not inhibit the activation of purified pro-MMP-9 by MMP-3 in vitro (FIG. 3H).

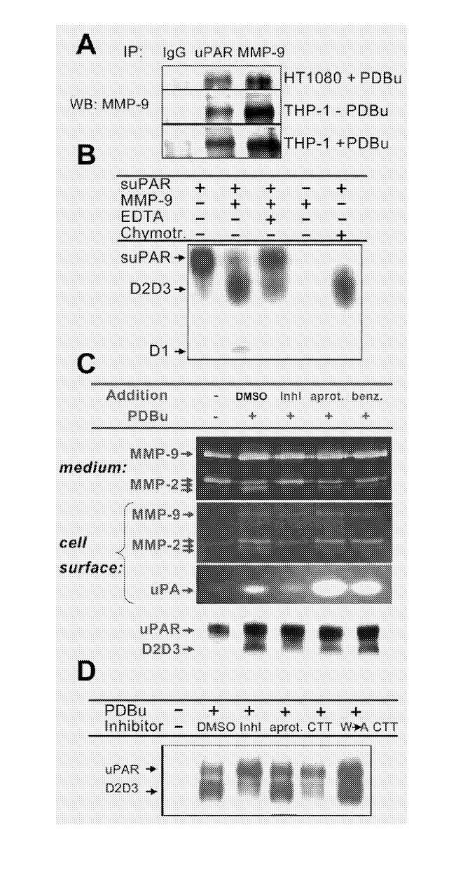

As the plasminogen activation cascade is involved in pro-MMP-9 activation, we considered the possibility that the urokinase receptor associates with MMP-9. Immunoprecipitations from PDBu-activated HT1080 cells showed that pro-MMP-9 co-precipitated with anti-uPAR antibodies, but not with the control antibodies (FIG. 4A). The association of uPAR and pro-MMP-9 was similarly found in THP-1 cells and was not affected by prior PDBu activation (FIG. 4A). Several proteinases are able to cleave uPAR (26,27), we thus asked whether also MMP-9 does so. Using purified proteins, we observed that MMP-9 cleaved the domain 1 (D1) from uPAR similarly as chymotrypsin does (FIG. 4B). The uPAR cleavage by MMP-9 occurred in the presence of aprotinin and was inhibited by the metalloproteinase inhibitor EDTA. uPAR cleavage occurs on the surface of phorbol-ester activated cells (26). To study the contribution of gelatinases in this process, we incubated HT1080 cells with proteinase inhibitors and analyzed the membrane protein-enriched lysates by western blotting with antibodies to uPAR. The gelatinase-selective inhibitor InhI, but not the serine proteinase inhibitors aprotinin or benzamidine, inhibited uPAR cleavage (FIG. 4C). The inhibition of uPAR cleavage was accompanied with reduced gelatinase levels in the conditioned medium and on the cell surface. In the conditioned medium, MMP-9 occurred in higher levels than MMP-2 whereas the opposite was true for the cell surface. The cell surface-bound MMP-9 was in the latent form as previously observed (28). In addition, the level of cell surface-bound uPA was reduced in the presence of InhI. uPAR cleavage on the THP-1 cells was similarly inhibited by InhI and CTT, but not by the inactive W→A CTT mutant peptide (15) or aprotinin (FIG. 4D). In the absence of PDBu, the THP-1 cells cultured in a serum-free medium expressed hardly detectable levels of uPAR.

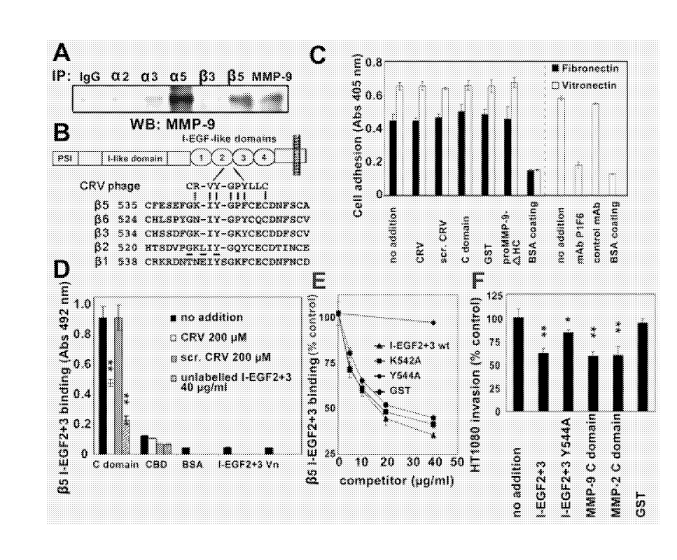

The CRV peptide is a mimic of an integrin β chain epitope—In non-leukocytic cells, uPAR is able to associate with β₁, β₃ and β₅ integrins (29-32). We thus investigated which integrin(s) could interact with MMP-9 in HT1080 cells. Immunoprecipitations were performed with antibodies against β₂, β₃, β₅, β₃ and β₅ integrins. Pro-MMP-9 associated with the α₅ and β₅ integrins indicating that α₅β₁ and α_{V}β₅ are the major integrins involved in pro-MMP-9 binding in HT1080 cells grown on a tissue culture-treated plastic (FIG. 5A). MMP-1 and -2 can interact with integrins through their C-terminal domains (6,33). Interestingly, a database search revealed that the CRV peptide bears a similarity to sequences found in the stalk of the integrin β chains, in particular the β₅ chain. Seven of the CRV amino acid residues had a matching or a similar residue in the β₅ sequence (FIG. 5B). These sequences are located in the cysteine-rich integrin-epidermal growth factor-like domain 2 (I-EGF2) and become exposed in the activated integrins as shown by the reactivity of activation state-specific antibodies (34,35). Indeed, the antibody KIM127 epitope maps to the CRV-like sequence in the β₅ integrin chain (34). To study whether MMP-9 binds to this integrin activation epitope, we first assessed the effect of the MMP-9 C domain on cell adhesion to vitronectin and fibronectin. Neither the C domain nor the pro-MMP-9-ΔHC (40 μg/ml) or the CRV peptide (200 μM) inhibited HT1080 cell adhesion to vitronectin or fibronectin (FIG. 5C). Adhesion to vitronectin occurred in a α_{V}β₅-dependent manner as demonstrated by inhibition with the α_{V}β₅ integrin-blocking antibody P1F6 (25 μg/ml). We did not observe specific adhesion of HT1080 cells to the immobilized C domain (not shown). These results indicated that the putative interaction site of the MMP-9 C domain in α₅β₁ and α_{V}β₅ is not the major RGD ligand-binding site or a cell adhesion determinant. This prompted us to express the I-EGF domains 2 and 3 (36) from the β₅ integrin. Interestingly, biotinylated β₅ I-EGF2+3 protein specifically bound to the MMP-9 C domain in a CRV-peptide inhibitable manner (FIG. 5D). The β₅ I-EGF2+3 fragment did not bind to MMP-9 CBD, vitronectin or itself (FIG. 5D) or the C domain of MMP-2 (not shown). The binding was cation independent (not shown) and could be inhibited with unlabelled β₅ I-EGF2+3. We next mutated the K542 and Y544 residues of the β₅ I-EGF2+3 to alanines to study the importance of the CRV-like sequence. This resulted in a decrease of activity, the K542A and Y544A proteins competing less efficiently for the binding of biotinylated β₅ I-EGF2+3 to the MMP-9 C domain (FIG. 5E). The Y544A mutation also decreased the ability of β₅ I-EGF2+3 to inhibit HT1080 invasion through matrigel (FIG. 5F). β₅ I-EGF2+3, MMP-9 C domain and MMP-2 C domain each inhibited HT1080 invasion with a similar potency, whereas GST had no effect.

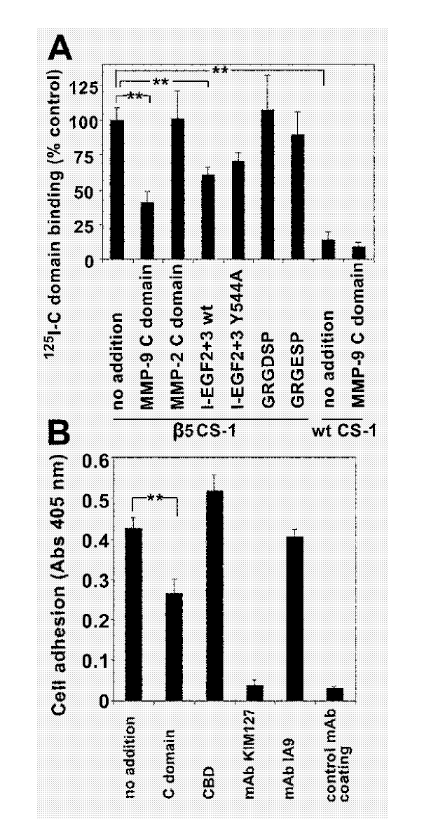

To find further evidence for the MMP-β₅ integrin interaction, we studied the binding of MMP-9 C domain to α_{V}β₅ expressing cells. ¹²⁵Iodine-labelled MMP-9 C domain showed a specific binding to β₅ integrin-transfected, but not to the untransfected CS-1 melanoma cells (FIG. 6A). The binding was competed with unlabelled MMP-9 C domain, the β₅ I-EGF2+3 fragment and to a lesser extent with the β₅ I-EGF2+3 Y544A mutant. No competition was observed with the MMP-2 C domain or the GRGDSP peptide.

To test if the MMP-9 C domain is able to bind to the CRV-like site of the β₂ integrin, we examined THP-1 cell binding to the immobilized KIM127 antibody. THP-1 cells bound to the KIM127 antibody, but not to an anti-His6-tag antibody (FIG. 5F). The C domain (50 μg/ml) inhibited the cell binding by 40%, whereas CBD did not (FIG. 5F). The specificity of the binding is shown by competition with soluble KIM127, but not by a control antibody. The C domain had no effect on THP-1 binding to another β₂ integrin-activating antibody R3F9C (not shown).

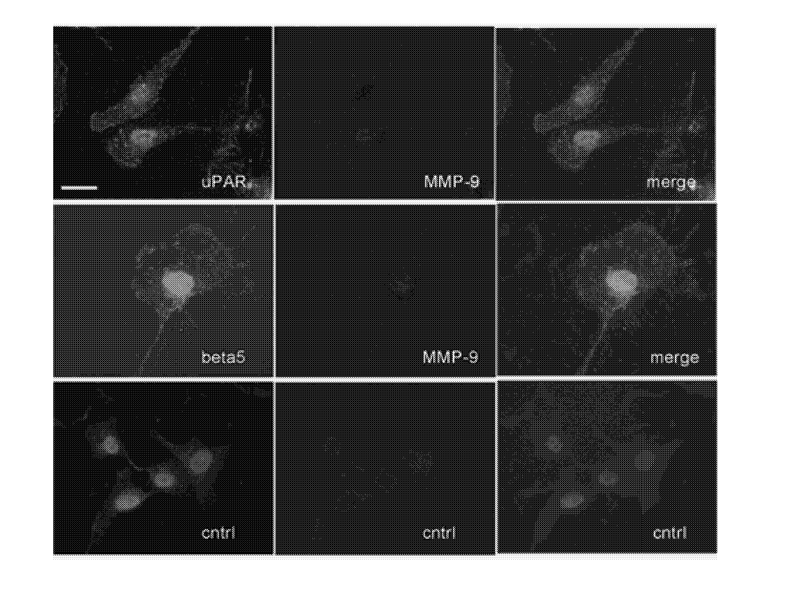

Double immunofluorescence stainings of HT1080 cells on vitronectin showed a partial colocalization for uPAR, MMP-9 and β₅ integrin. MMP-9 was concentrated on the leading edge of the cells where the colocalization with integrin and uPAR are evident (FIG. 7). Only non-specific nuclear staining was observed with irrelevant control antibodies. Colocalization of uPAR with MMP-9 was also found on the surface of THP-1 cells (not shown).

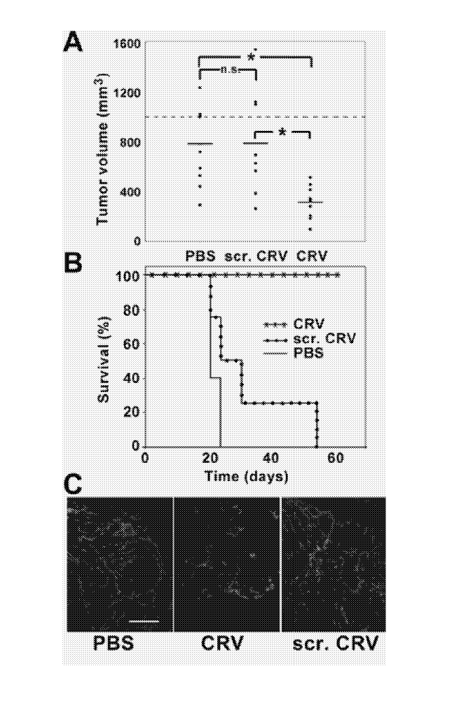

As a final test of reactivity of the CRV peptide, we assessed its effect on tumor growth in vivo. Mice carrying HSC-3 tongue squamous cell carcinoma xenografts were treated with the peptide when the subcutaneous tumors were in an early phase and not yet visible. CRV, the scrambled peptide or PBS were injected intravenously five times. At 31 days, a statistically significant inhibition of tumor growth by CRV was observed in comparison to the scrambled peptide or PBS (FIG. 8A). CRV increased the survival of the mice and after two months all five CRV-injected mice were alive, whereas the mice given the scrambled peptide or PBS had been euthanized due to large tumors (FIG. 8B). The effect of CRV could at least partially be accounted for inhibition of angiogenesis. The CRV-treated mice had a less developed tumor vasculature as shown by immunostaining of the endothelial marker CD31 (FIG. 8C).

Discussion

We have developed domain-specific peptide probes to the gelatinases and examined molecular interactions important for these enzymes. Each of the domain-specific peptides inhibited cell migration indicating that the three major domains of MMP-9, the catalytic domain, CBD and the C domain each play a distinct role. We have previously shown that in leukocytes pro-MMP-9 interacts with the α_{M} and α_{L} integrin I domains through the catalytic domain (12). Here we have found another integrin interaction for MMP-9, where the C domain of MMP-9 binds to the integrin β subunit. In contrast to the I domain interaction which occurs in the presence of calcium and presumably maintains pro-MMP-9 inactive, the C domain/β subunit interaction requires activated integrins and appears to play a dynamic role in mediating MMP-9 activation and pericellular gelatinolysis.

Of the MMP-9 binding peptides identified in this study, the CBD-binding peptide PPC functioned as an exosite inhibitor of MMP-2 and -9 inhibiting gelatin binding and degradation but had no inhibitory effect on the MMP-9 interactions with integrins. We identified a PPC-like sequence in the heparin-binding domain of fibronectin as a CBD recognition site. Vitronectin had a similar, but apparently lower affinity binding-site for CBD. As PPC did not bind to the fibronectin type II repeats of fibronectin, it could serve as a lead compound for the development of highly specific gelatinase inhibitors.

The C-terminal domain-binding CRV peptide did not affect the enzymatic activity of MMP-9, but inhibited dimerization of the MMP-9 C domain, activation of the pro-MMP-9 via plasminogen/MMP-3 dependent pathway, and pericellular gelatinolysis. Several findings indicate that CRV is a mimick of the activation epitope in the integrin β subunit, preferentially the β₅ subunit. The C domain of MMP-9 inhibited leukocyte adhesion to the KIM127 antibody, which recognizes the CRV homologous site in the β₂ integrin. The recombinant β₅ integrin I-EGF2+3 fragment specifically bound to the C domain in a CRV-dependent manner and the single alanine mutations of the ⁵⁴²K and ⁵⁴⁴Y residues in the β₅ I-EGF2+3 decreased its activity. The β₅ integrin-transfected cells, but not the untransfected cells bound the C domain of MMP-9. In HT1080 cells, pro-MMP-9 was co-precipitated with antibodies to β₅ integrins, and the β₅ I-EGF2+3 fragment and the C domain both inhibited invasiveness of this cell line. MMP-9 and β₅ integrins similarly localized to the leading edge of the HT1080 cells. However, we cannot exclude the possibility that the CRV peptide inhibits also other C domain-mediated interactions.

We did not observe association of MMP-9 with α_{V}β₃ in HT1080 cells although a functional linkage between MMP-9 and the active α_{V}β₃ integrin has been found (37). This may reflect that fact that HT1080 cells utilize the α_{V}β₅ integrin for vitronectin adhesion. MMP-9 binding to α_{V}β₅ may be physiologically more relevant as α_{V}β₅ and MMP-9 expression are under similar transcriptional regulation (8,38). In turn, α_{V}β₃ and MMP-2 appear to be co-regulated (39). In our studies the CRV peptide only weakly inhibited pro-MMP-2 activation and the C domains of MMP-2 and MMP-9 did not compete with each other in binding assays.

The finding that CRV mimicks an integrin activation epitope provides an explanation for the requirement of ligand-engaged integrins in pro-MMP-9 activation (37,40). We also demonstrate, that uPAR, which is required for MMP-9 activation associates with pro-MMP-9 in HT1080 and THP-1 cells. uPAR was a substrate for MMP-9 in vitro and the cellular cleavage of uPAR was gelatinase dependent. Cleavage by MMP-9 resulted in the release of the D1 domain of uPAR, which has also been observed with other MMPs such as MMP-12 (27). Functionally, uPAR cleavage causes loss of uPA binding and the dissociation of uPAR and integrins (41). Thus, MMP-9 not only regulates its own activation but also uPAR function. Interestingly, co-operation of MMP-9 and uPAR has been shown to be essential for the intravasation of tumor cells (42). Also, uPA/uPAR and gelatinases co-exist in transport vesicles in migrating cells (43,44).

Inhibition of tumor growth by CRV suggests an important function for the MMP-9/α_{V}β₅ pair in primary tumor growth and/or angiogenesis. However, increased tumor growth rather than inhibition is observed in both the β₃ and β₅ integrin knockout mice (45) and also in mice with low plasma levels of MMP-9 (46). The ability of MMP-9 to generate angiostatin or tumstatin (47) may explain these contradictory findings, and perhaps α_{V}β₅-bound MMP-9 is also used for angiostatin generation. Furthermore, the cleavage of uPAR by MMP-9 could also inhibit tumor spreading. As tumor therapies aimed at direct inhibition of MMP activity have not been very successful, the noncatalytic means to inhibit MMPs may be more attractive (6). It is encouraging that our phage display-developed peptides specifically interfere with different integrin-mediated interactions blocking either the MMP catalytic or the C-terminal domain binding, suggesting that specific drugs can be developed that locally prevent gelatinase function, but not the enzymatic activity. Supporting this conception, also the β₂-integrin ligand DDGW peptide, which blocks the α_{M}β₂ integrin/pro-MMP-9 complex, is active in vivo inhibiting neutrophil recruitment in an acute inflammation model in mice (48).

Our model of the MMP-9 interactions with integrins is based on a “peptidoscopic” view obtained with phage display peptides and suggests that pro-MMP-9 can interact with integrins in two ways. In leukocytes the interaction between the integrin I domain and the MMP-9 catalytic domain is dominant and apparently keeps pro-MMP-9 in an inactive form. Ligand binding activates the integrin and exposes the activation epitope in the β chain, which can act as a docking site for the C domain of MMP-9. MMP-9 may then be activated by proteases or becomes catalytically competent by direct binding to a substrate (49). In integrins that lack an I domain in the α subunit, the MMP-9 C domain-directed interaction may be the dominant interacting site.

TABLE 1
Pro-MMP-9 binding peptide sequences

| Group I: | Group II: |
|---|---|
| CBD-binding sequences | C domain-binding sequences |
| CGYGRFSPPC^{a} (6) | CRVYGPYLLC |
| CGWGRYSPPC | CRWYGPILWC (2) |
| CGFGRWQPPC | CRWYGPWALC |
| FN TPNSLLVSWQPPRARIT | CRWYGPWVWC |
| VN PETLHPGRPQPPAEEEL | CRFYGAWLLC |
| | CRHYGPFSIC |
| | CRRYGPFMVC |
| | CRTYGWWVVC |
| | CRYYGWLTVC |
| | CKWYGLFQLC |
| | CHSYGPFVVC |
| | CNWYGWFRVC |

^{a}The residues underlined are the same as in fibronectin (FN) and vitronectin (VN). The number of isolated phage is indicated in brackets.

### REFERENCES

1. Sternlicht, M. D., and Werb, Z. (2001) Annu. Rev. Cell Dev. Biol. 17, 463-516.
2. Koivunen, E., Arap, W., Valtanen, H., Rainisalo, A., Medina, O. P., Heikkila, P., Kantor, C., Gahmberg, C. G., Salo, T., Konttinen, Y. T., Sorsa, T., Ruoslahti, E., and Pasqualini, R. (1999) Nat. Biotechnol. 17, 768-774.
3. Tamura, Y., Watanabe, F., Nakatani, T., Yasui, K., Fuji, M., Komurasaki, T., Tsuzuki, H., Maekawa, R., Yoshioka, T., Kawada, K., Sugita, K., and Ohtani, M. (1998) J. Med. Chem. 41, 640-649.
4. Overall, C. M. (2002) Mol. Biotechnol. 22, 51-86.
5. Cha, H., Kopetzki, E., Huber, R., Lanzendorfer, M., and Brandstetter, H. (2002) J. Mol. Biol. 320, 1065-1079.
6. Brooks, P. C., Silletti, S., von Schalscha, T. L., Friedlander, M., and Cheresh, D. A. (1998) Cell 92, 391-400.
7. Deryugina, E. I., Ratnikov, B., Monosov, E., Postnova, T. I., DiScipio, R., Smith, J. W., and Strongin, A. Y. (2001) Exp. Cell Res. 263, 209-223.
8. Ramos-DeSimone, N., Hahn-Dantona, E., Sipley, J., Nagase, H., French, D. L., and Quigley, J. P. (1999) J. Biol. Chem. 274, 13066-13076.
9. Sorsa, T., Salo, T., Koivunen, E., Tyynela, J., Konttinen, Y. T., Bergmann, U., Tuuttila, A., Niemi, E., Teronen, O., Heikkila, P., Tschesche, H., Leinonen, J., Osman, S., and Stenman, U. H. (1997) J. Biol. Chem. 272, 21067-21074.
10. Wang, X. Q., Sun, P., and Paller, A. S. (2003) J. Biol. Chem. 30, 30
11. Fridman, R., Toth, M., Chvyrkova, I., Meroueh, S. O., and Mobashery, S. (2003) Cancer Metastasis Rev. 22, 153-166.
12. Stefanidakis, M., Bjorklund, M., Ihanus, E., Gahmberg, C. G., and Koivunen, E. (2003) J. Biol. Chem. 278, 34674-34684.
13. Koivunen, E., Restel, B. H., Rajotte, D., Lahdenranta, J., Hagedorn, M., Arap, W., and Pasqualini, R. (1999) Methods Mol. Biol. 129, 3-17.
14. Koivunen, E., Ranta, T. M., Annila, A., Taube, S., Uppala, A., Jokinen, M., van Willigen, G., Ihanus, E., and Gahmberg, C. G. (2001) J. Cell Biol. 153, 905-916.
15. Bjorklund, M., Valtanen, H., Savilahti, H., and Koivunen, E. (2003) Comb. Chem. High Throughput Screen. 6, 29-35.
16. Bjorklund, M., and Koivunen, E. (2004) Letters in Drug Design &Discovery 1, 163-167
17. Yatohgo, T., Izumi, M., Kashiwagi, H., and Hayashi, M. (1988) Cell Struct. Funct. 13, 281-292.
18. Nyberg, P., Heikkila, P., Sorsa, T., Luostarinen, J., Heljasvaara, R., Stenman, U. H., Pihlajaniemi, T., and Salo, T. (2003) J. Biol. Chem. 278, 22404-22411.
19. Filardo, E. J., Deming, S. L., and Cheresh, D. A. (1996) J. Cell Sci. 109, 1615-1622.
20. Vogel, B. E., Lee, S. J., Hildebrand, A., Craig, W., Pierschbacher, M. D., Wong-Staal, F., and Ruoslahti, E. (1993) J. Cell Biol. 121, 461-468.
21. Pasqualini, R., Bodorova, J., Ye, S., and Hemler, M. E. (1993) J. Cell Sci. 105, 101-111.
22. Behrendt, N., Ploug, M., Ronne, E., Hoyer-Hansen, G., and Dano, K. (1993) Methods Enzymol. 223, 207-222.
23. Myohanen, H., Virtanen, I., and Vaheri, A. (2001) Biol. Chem. 382, 1563-1573.
24. Giordano, R. J., Cardo-Vila, M., Lahdenranta, J., Pasqualini, R., and Arap, W. (2001) Nat. Med. 7, 1249-1253.
25. Trexler, M., Briknarova, K., Gehrmann, M., Llinas, M., and Patthy, L. (2003) J. Biol. Chem. 278, 12241-12246.
26. Koolwijk, P., Sidenius, N., Peters, E., Sier, C. F., Hanemaaijer, R., Blasi, F., and van Hinsbergh, V. W. (2001) Blood 97, 3123-3131.
27. Andolfo, A., English, W. R., Resnati, M., Murphy, G., Blasi, F., and Sidenius, N. (2002) Thromb. Haemost. 88, 298-306.
28. Toth, M., Gervasi, D. C., and Fridman, R. (1997) Cancer Res. 57, 3159-3167.
29. Simon, D. I., Wei, Y., Zhang, L., Rao, N. K., Xu, H., Chen, Z., Liu, Q., Rosenberg, S., and Chapman, H. A. (2000) J. Biol. Chem. 275, 10228-10234.
30. Xue, W., Mizukami, I., Todd, R. F., 3rd, and Petty, H. R. (1997) Cancer Res. 57, 1682-1689.
31. Carriero, M. V., Del Vecchio, S., Capozzoli, M., Franco, P., Fontana, L., Zannetti, A., Botti, G., D'Aiuto, G., Salvatore, M., and Stoppelli, M. P. (1999) Cancer Res. 59, 5307-5314.
32. Aguirre Ghiso, J. A., Kovalski, K., and Ossowski, L. (1999) J. Cell Biol. 147, 89-104.
33. Stricker, T. P., Dumin, J. A., Dickeson, S. K., Chung, L., Nagase, H., Parks, W. C., and Santoro, S. A. (2001) J. Biol. Chem. 276, 29375-29381.
34. Lu, C., Ferzly, M., Takagi, J., and Springer, T. A. (2001) J. Immunol. 166, 5629-5637.
35. Bazzoni, G., Shih, D. T., Buck, C. A., and Hemler, M. E. (1995) J. Biol. Chem. 270, 25570-25577.
36. Beglova, N., Blacklow, S. C., Takagi, J., and Springer, T. A. (2002) Nat. Struct. Biol. 9, 282-287.
37. Rolli, M., Fransvea, E., Pilch, J., Saven, A., and Felding-Habermann, B. (2003) Proc. Natl. Acad. Sci. USA 21, 21
38. Friedlander, M., Brooks, P. C., Shaffer, R. W., Kincaid, C. M., Varner, J. A., and Cheresh, D. A. (1995) Science 270, 1500-1502.
39. Lyden, D., Young, A. Z., Zagzag, D., Yan, W., Gerald, W., O'Reilly, R., Bader, B. L., Hynes, R. O., Zhuang, Y., Manova, K., and Benezra, R. (1999) Nature 401, 670-677.
40. Festuccia, C., Angelucci, A., Gravina, G., Eleuterio, E., Vicentini, C., and Bologna, M. (2002) Exp. Cell Res. 280, 1-11.
41. Montuori, N., Carriero, M. V., Salzano, S., Rossi, G., and Ragno, P. (2002) J. Biol. Chem. 277, 46932-46939.
42. Kim, J., Yu, W., Kovalski, K., and Ossowski, L. (1998) Cell 94, 353-362.
43. Ginestra, A., Monea, S., Seghezzi, G., Dolo, V., Nagase, H., Mignatti, P., and Vittorelli, M. L. (1997) J. Biol. Chem. 272, 17216-17222.
44. Pedersen, T. L., Plesner, T., Horn, T., Hoyer-Hansen, G., Sorensen, S., and Hansen, N. E. (2000) Ultrastruct. Pathol. 24, 175-182.
45. Reynolds, L. E., Wyder, L., Lively, J. C., Taverna, D., Robinson, S. D., Huang, X., Sheppard, D., Hynes, R. O., and Hodivala-Dilke, K. M. (2002) Nat. Med. 8, 27-34.
46. Pozzi, A., LeVine, W. F., and Gardner, H. A. (2002) Oncogene 21, 272-281.
47. Hamano, Y., Zeisberg, M., Sugimoto, H., Lively, J. C., Maeshima, Y., Yang, C., Hynes, R. O., Werb, Z., Sudhakar, A., and Kalluri, R. (2003) Cancer Cell 3, 589-601.
48. Stefanidakis, M., Ruohtula, T., Borregaard, N., Gahmberg, C., and Koivunen, E. (in press) J. Immunol.
49. Bannikov, G. A., Karelina, T. V., Collier, I. E., Marmer, B. L., and Goldberg, G. I. (2002) J. Biol. Chem. 277, 16022-16027.

1. A matrix metalloproteinase inhibitor and down-regulator comprising the structure of the peptide motif
CRXYGPXXXC (SEQ ID NO: 3)
wherein X is any amino acid residue.

2. The matrix metalloproteinase inhibitor and down-regulator according to claim 1 wherein the peptide motif is CRVYGPYLLC (SEQ ID NO: 7).

3. A pharmaceutical composition comprising a matrix metalloproteinase inhibitor and down-regulator according to claim 1 and a pharmaceutically acceptable carrier.

4. The use of a matrix metalloproteinase inhibitor and down-regulator according to claim 1 for the manufacture of a pharmaceutical composition for the treatment of matrix metalloproteinase (MMP) dependent conditions.

5. The use according to claim 4 for the manufacture of a pharmaceutical composition for the treatment of conditions dependent on MMP-2 and/or MMP-9.

6. A process for the preparation of a matrix metalloproteinase inhibitor/down-regulator according to claim 1, which process comprises solid-phase Merrifield peptide synthesis.

7. A method for the therapeutic or prophylactic treatment of matrix metalloproteinase dependent conditions in mammals comprising administering to said mammal a matrix metalloproteinase inhibitor/down-regulator according to claim 1 in an amount which is effective in inhibiting and down-regulating MMP activations, expressions and/or functions in said mammal.

8. The method according to claim 7 for the therapeutic or prophylactic treatment of conditions dependent on MMP-2 and/or MMP-9.

9. A method for inhibiting the activations, expressions, functions and actions of matrix metalloproteinases in mammals, comprising administering to said mammal a matrix metalloproteinase inhibitor and down-regulator according to claim 1 in an amount which is effective in blocking the activities, activations and actions of MMPs.

10. The method according to claim 9 for inhibiting the expressions, activations and actions of MMP-2 and/or MMP-9.

11. A method for inhibiting and down-regulating matrix metalloproteinases in vitro comprising adding to an in vitro system a matrix metalloproteinase inhibitor and down-regulator according to claim 1 in an amount which is effective in inhibiting and down-regulating the MMP activity.

12. The method according to claim 11 wherein the matrix metalloproteinases to be inhibited and down-regulated are MMP-2 and/or MMP-9.

13. The use of a matrix metalloproteinase inhibitor and down-regulator according to claim 1 in biochemical isolation and purification procedures of matrix metalloproteinases.

14. Use of matrix metalloproteinase inhibitor and down-regulator according to claim 1 in improving targeting of liposomes to tumor cells.

15. Use according to claim 14, wherein the tumor cells are tumor cells expressing matrix metalloproteinases.
